(19) United States
(12) Patent Application Publication
(10) Pub. No.: US 2013/0053834 A1
(43) Pub. Date: Feb. 28, 2013

(54) INSTRUMENT FOR MANIPULATING SURGICAL IMPLANTS

(71) Applicants: Nathan Meyer, Carlsbad, CA (US); Anand Parikh, San Diego, CA (US)

(72) Inventors: Nathan Meyer, Carlsbad, CA (US); Anand Parikh, San Diego, CA (US)

(73) Assignee: Alphatec Spine, Inc., Carlsbad, CA (US)

(21) Appl. No.: 13/589,964

(22) Filed: Aug. 20, 2012

Related U.S. Application Data
(60) Provisional application No. 61/528,819, filed on Aug. 30, 2011.

(51) Int. Cl. A61B 17/00

(52) U.S. Cl. 606/1

(57) ABSTRACT

An instrument for grasping and articulating a surgical implant includes a first arm having a distal end with a proximal-facing surface and a second arm pivotably coupled to the first arm and having a distal end with a distal-facing surface. The proximal-facing surface and the distal-facing surface grasp the implant at a first angle in a first position and a second angle in a second position.

### CROSS REFERENCE TO RELATED APPLICATIONS

The present application claims priority to U.S. Provisional Application 61/528,819, filed on Aug. 30, 2011, entitled “Instrument for Manipulating Surgical Implants” which is incorporated by reference in its entirety herein

### FIELD

The present disclosure generally relates to the field of surgery, and more particularly to an instrument for grasping and articulating a surgical implant.

### BACKGROUND

Various surgeries require the implantation of surgical devices to reinforce deficient bone structure in the body. For example, surgical implants have been used in neurosurgical procedures to treat curvature, trauma, deformity, and degenerative conditions of the spine. These implants typically include systems of screws and rods placed bilaterally along the vertebrae and spacers placed between vertebrae. Additional rods may further be used as cross-link members that bridge the sagittal of the spine. The screws and rods and spacers are capable of supporting alignment of the vertebrae.

Surgical procedures for implanting the rods require much care. In particular, minimally invasive surgery (MIS) may require the manipulation of the rods so as to not damage the spinal cord, other nerves associated therewith, and surrounding tissue. For example, in a typical MIS procedure, surgical openings may only be large enough to insert the screws and associated screw extenders. The screw extenders may couple to heads of the screws and extend through the surgical openings outside the body. These screw extenders may be slotted to accommodate insertion of the rods.

Typically, the rod must be inserted at a nearly vertical angle corresponding to the anteroposterior axis of the body in order to pass through the slot in the screw extender and the surgical opening. Similarly, an insertion instrument that grasps the rod must be configured to pass through the slot and the surgical opening. Once the rod has passed through the surgical opening, the rod must be positioned at a nearly horizontal angle corresponding to the transverse axis of the body in order to attach to an adjacent screw head. Thus, the insertion instrument must be capable of adjusting the angle of the rod and positioning the rod within the heads of the screws.

Existing rod insertion instruments require varying amounts of delicate manipulation and gripping forces to successfully grasp and position the rod within a surgical area. Many instruments include various and complex moving parts. Furthermore, many instruments with pins, latches, and connecting elements require mating features on the rod such as holes, detents, and/or grooves to permit proper connection. Often times, these additional features are located on a particular end of the rod, and thus, the instrument may only grasp the rod at that particular end.

Accordingly, it is an object of the present invention to provide an instrument which firmly grasps and reliably manipulates implant rods with delicate precision and without exorbitant force required by the user. It is another object of the present invention to provide an instrument with reduced complexity to decrease the likelihood of instrument failure. It is yet another object of the present invention to provide an instrument which may grasp any rod at any location along the length of the rod.

### SUMMARY

An instrument for grasping and articulating a surgical implant includes a first arm having a distal end with a proximal-facing surface and a second arm pivotably coupled to the first arm and having a distal end with a distal-facing surface. The proximal-facing surface and the distal-facing surface grasp the implant at a first angle in a first position and a second angle in a second position.

In other features, a first portion of the proximal-facing surface opposes a first portion of the distal-facing surface in the first position and a second portion of the proximal-facing surface opposes a second portion of the distal facing surface in the second position. In still other features, at least one of the first arm and the second arm includes a first length in the first position and a second length in the second position. In yet other features, the first angle is greater than sixty degrees from a longitudinal axis of the instrument and the second angle is less than sixty degrees from the longitudinal axis of the instrument. In still other features, the distal ends offset in the first position and align in the second position. In yet other features, the proximal-facing surface and the distal-facing surface are separated by a fixed distance in the first and second positions that corresponds to a thickness of the implant.

In still other features, the first arm includes an adjustable length and the second arm includes a fixed length greater than the adjustable length. In still other features, the adjustable length decreases from a first length to a second length as the first arm and the second arm pivot from the first position to the second position.

In still other features, the first arm includes a fixed length and the second arm includes an adjustable length that is greater than the fixed length. In still other features, the adjustable length increases from a first length to a second length as the first arm and the second arm pivot from the first position to the second position.

In one example, an instrument for gripping and manipulating a spinal rod includes a fixed length arm having a distal end with first and second proximal-facing grips and an adjustable length arm having a distal end with first and second distal-facing grips and pivotably coupled and movable between first and second positions relative to the fixed length arm. The adjustable length arm includes a first length and the first proximal-facing grip and the first distal-facing grip grasp the spinal rod at a first angle in the first position. The adjustable length arm includes a second length and the second proximal-facing grip and the second distal-facing grip grasp the spinal rod at a second angle in the second position.

In other features, the first length is greater than the second length. In still other features, the fixed length arm includes a fixed length greater than the first and second lengths. In yet other features, the distal ends offset in the first position and align in the second position. In yet other features, a bias member biases the adjustable length arm to the first length in the first position and compresses when the adjustable length arm decreases to the second length in the second position.

In another example, an instrument for gripping and manipulating a spinal rod includes a fixed length arm having a distal end with first and second distal-facing grips and an adjustable length arm having a distal end with first and second proximal-facing grips and pivotably coupled and movable between first and second positions relative to the fixed length arm. The adjustable length arm includes a first length and the first proximal-facing grip and the first distal-facing grip grasp the spinal rod at a first angle in the first position. The adjustable length arm includes a second length and the second proximal-facing grip and the second distal-facing grip grasp the spinal rod at a second angle in the second position.

In other features, the first length is less than the second length. In still other features, the fixed length arm includes a fixed length less than the first and second lengths. In yet other features, the distal ends offset in the first position and align in the second position. In yet other features, a bias member biases the adjustable length arm to the first length in the first position and stretches when the adjustable length arm increases to the second length in the second position.

### BRIEF DESCRIPTION OF THE DRAWINGS

FIG. 1 is an elevational side view of an exemplary instrument for grasping and articulating a surgical implant according to the principles of the present disclosure.

FIG. 2 is an elevational side view of the instrument grasping a surgical implant at a first angle in a first position according to the principles of the present disclosure.

FIG. 3 is an elevational side view of the instrument grasping the surgical implant at a second angle in a second position according to the principles of the present disclosure.

FIG. 4 is an elevational side view of another exemplary instrument for grasping and articulating a surgical implant according to the principles of the present disclosure.

FIG. 5 is an elevational side view of the instrument grasping the surgical implant at a first angle in a first position according to the principles of the present disclosure.

FIG. 6 is an elevational side view of the instrument grasping the surgical implant at a second angle in a second position according to the principles of the present disclosure.

FIGS. 7-9 are magnified side views of the distal end of the instruments in FIGS. 1-3 and FIGS. 4-6 according to the principles of the present disclosure.

### DETAILED DESCRIPTION

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The instrument of the present invention includes various features that enable grasping and articulation of a surgical implant relative to a surgical area. For example, the instrument may grasp and articulate a spinal rod for attachment to pedicle screws in a minimally invasive surgical procedure. The instrument includes a first arm with a distal end that includes a proximal-facing surface and a second arm pivotably coupled to the first arm with a distal end that includes a distal-facing surface. The instrument may be maneuvered between a first position and a second position by rotating the second arm relative to the first arm in a scissors-action. The proximal-facing surface and the distal-facing surface grasp the rod at a first angle in the first position and a second angle in the second position.

In some examples, the proximal-facing surface may include first and second portions and the distal-facing surface may include first and second portions. At least one of the first and second arms may be an adjustable length arm that includes a variable length that may change between the first and second positions. In the first position, the first portion of the proximal-facing surface may oppose the first portion of the distal-facing surface. The variable length of the adjustable length arm may include a first length such that the first portions may be separated by the thickness of the rod. In the second position, the second portion of the proximal-facing surface may oppose the second portion of the distal-facing surface. The variable length of the adjustable length arm may include a second length such that the second portions may be separated by the thickness of the rod. As the instrument rotates from the first position to the second position, the proximal-facing and distal-facing surfaces may move the rod from a first angle to a second angle relative to the arms.

The instrument of the present invention is advantageous over the prior art for various reasons. The instrument reduces the amount of force required to grip the rod by increasing leverage on the distal ends. The instrument provides greater contact area between the instrument and the rod to increase stability and maneuverability. The instrument uses a simplified scissors-action to reduce complexity and decrease the likelihood of instrument failure. The instrument requires fewer features that interact with the rod and may grasp any rod at any location along the length of the rod. The instrument includes gripping surfaces that may grasp the rod at the ends or anywhere along the middle portion of the implant.

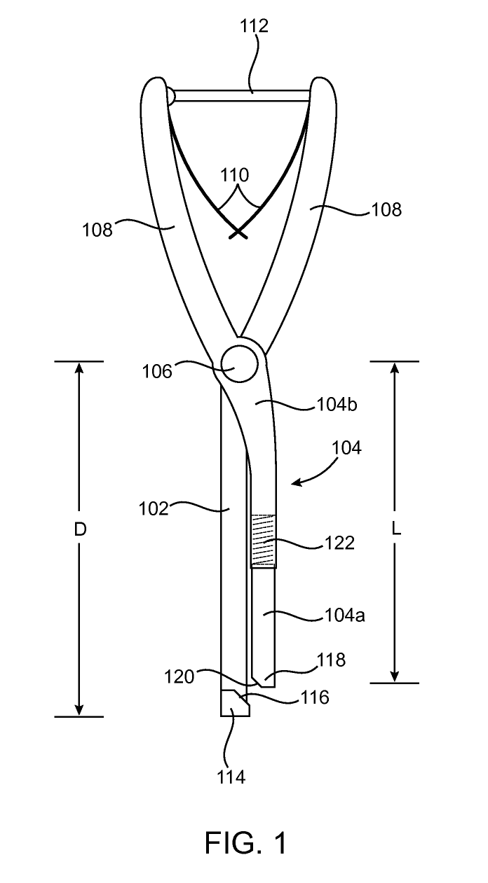

FIGS. 1-6 illustrate exemplary instruments 100 for grasping and articulating a surgical implant according to the principles of the present disclosure. Each instrument 100 may be, for example, a rod inserter that grasps a spinal rod 200 and articulates the rod 200 into heads of associated poly-axial screws (not shown) in a minimally invasive surgical procedure. The instrument 100 may include a first arm 102 pivotably coupled to second arm 104 at a pivot 106 near a midsection of the instrument 100. The pivot 106 may include any of a rivet, screw, bolt, or the like. The pivot 106 may include an adjustable pivot with slotted engagement features similar to a channel lock pliers to alter the pivot location (not shown). The instrument 100 may be movable between at least first and second positions by rotating the first arm 102 and second arm 104 relative to one another similar to a scissors or pliers. At least one of the arms includes an adjustable length L while the other one of the arms may include a fixed length D or an adjustable length L.

The instrument 100 and actuation of the instrument 100 may be described relative to a Cartesian coordinate system centered at the pivot 106. For example, a Z-axis may correspond to an axis of rotation about the pivot 106. A Y-axis may correspond to a centerline of the instrument 100. An X-axis may run perpendicular to the Y and Z axes. Thus, the first arm 102 may pivot relative to the second arm 104 about the Z-axis in a rotational plane XY defined by the X and Y axes.

The instrument 100 may include handle portions 108 extending proximally from the pivot 106 to enable actuation of the instrument 100 by a surgeon. For example, each of the arms 102 and 104 may be coupled to a corresponding handle portion 108. The instrument 100 may include additional features such as a handle spring 110 and a locking system 112 disposed between the handle portions 108. The handle spring 110 may, for example, include a pair of leaf springs that bias the handle portions 108 apart from one another. The locking system 112 may, for example, include a ratchet system with interlocking teeth to allow for locking the handles portions 108 at various positions including the first and second positions.

In various examples, the instrument 100 includes an adjustable length arm that changes length as the instrument 100 rotates between the first and second positions. For example, the distal ends of the first and second arms 102 and 104 may grasp a rod in the first position. With pressure on the handle portions 108, the distal ends of the arms are brought closer together. At least one of the arms changes length to accommodate the thickness of the rod. The other arm may include a fixed length or adjustable length as well.

In the example shown in FIGS. 1-3, the first arm 102 includes the fixed length D measurable from the pivot 106 to the distal end. At the distal end, a base portion 114 may partially project away from the first arm 102 in the Z direction and thus, break the rotational XY plane. The base portion 114 includes a proximal-facing surface 116 (hereinafter “proximal surface”) that faces towards the proximal end of the instrument 100. The second arm 104 includes the adjustable length L measurable from the pivot 106 to the distal end. At the distal end, a tip 118 includes a distal-facing surface 120 (hereinafter “distal surface”) that faces towards the distal end of the instrument 100. Each of the surfaces 116 and 120 may include a recessed or contoured portion configured to receive the rod 200. The surfaces may include a gripping material or coating to increase friction forces between the rod 200 and the instrument 100.

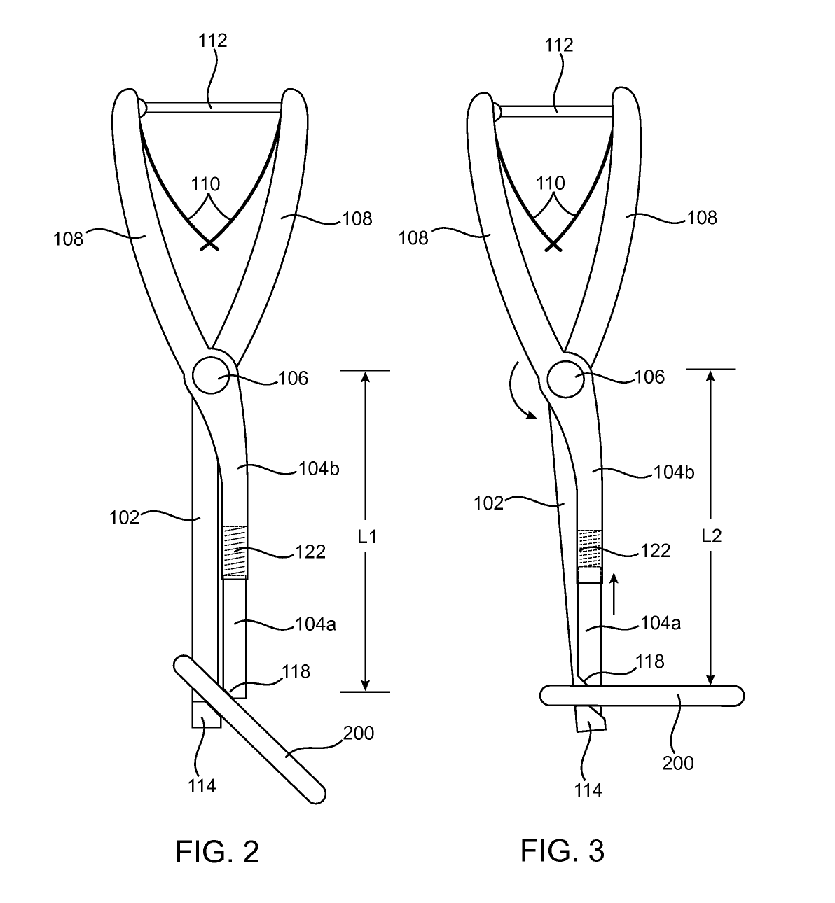

In the present example, the second arm 104 includes features that enable changes in the length L. For example, the second arm 104 may include a distal portion 104a that slides relative to a proximal portion 104b. A bias member 122, such as a spring, may couple the distal portion 104a to the proximal portion 104b. The bias member 122 may compresses and/or stretch between the distal portion 104a and proximal portion 104b. The bias member 122 may bias the second arm 104 to a first length L1 and compress or stretch as the second arm 104 decreases or increases to a second length L2. For example, the second arm 104 may include the first length L1 when the instrument 100 is in the first position as illustrated in FIG. 2 and decrease to a second length L2 when the instrument 100 is in the second position as illustrated in FIG. 3.

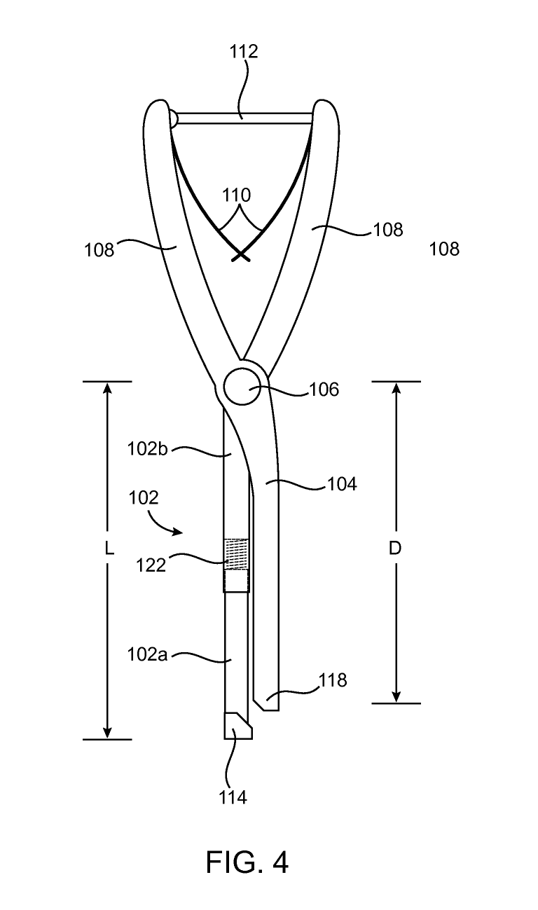

In the example shown in FIGS. 4-6, the first arm 102 includes the adjustable length L measurable from the pivot 106 to the distal end. At the distal end, the base portion 114 may partially project away from the first arm 102 in the Z direction and thus, break the rotational XY plane. The base portion 114 includes the proximal surface 116 that faces towards the proximal end of the instrument 100. The second arm 104 includes the fixed length D measurable from the pivot 106 to the distal end. At the distal end, the tip 118 includes the distal surface 120 that faces towards the distal end of the instrument 100. Each of the surfaces 116 and 120 may include a recessed or contoured portion configured to receive the rod 200. The surfaces may include a gripping material or coating to increase friction forces between the rod 200 and the instrument 100.

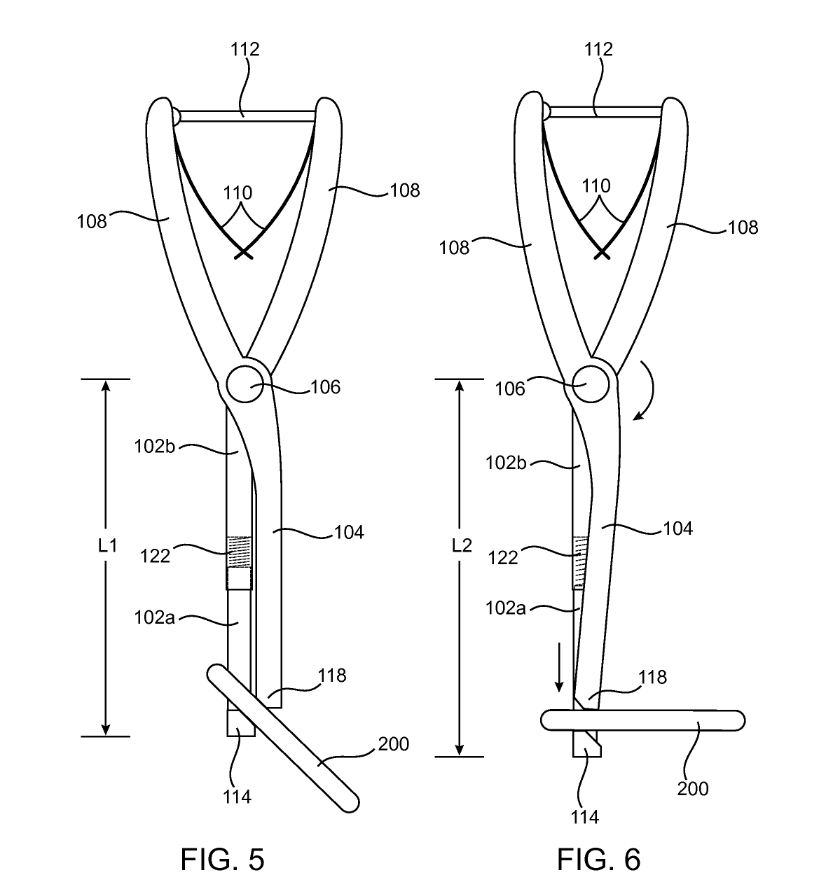

In the present example, the first arm 102 includes features that enable changes in the length L. For example, the first arm 102 may include a distal portion 102a that slides relative to a proximal portion 102b. The bias member 122, such as a spring, may couple the distal portion 102a to the proximal portion 102b. The bias member 122 may compresses and/or stretch between the distal portion 102a and proximal portion 102b. The bias member 122 may bias the first arm 104 to a first length L1 and compress or stretch as the first arm 102 decreases or increases to a second length L2. For example, the first arm 102 may include the first length L1 when the instrument 100 is in the first position as illustrated in FIG. 5 and increase to a second length L2 when the instrument 100 is in the second position as illustrated in FIG. 6.

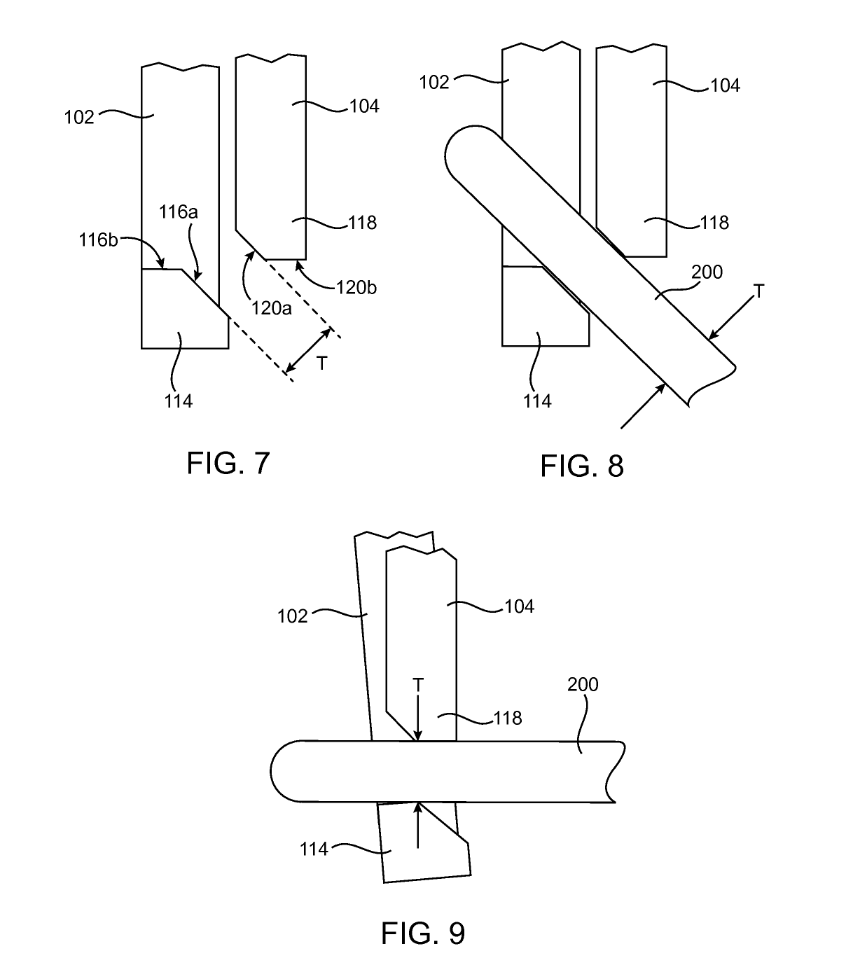

At the distal end of the instrument 100, shown in greater detail in FIG. 7, the arms 102 and 104 include gripping surfaces configured to grasp the rod 200. In various examples, the distal end includes at least one proximal surface 116 and at least one distal surface 120. The proximal surface 116 may include a continuously curved surface or distinctly angled portions such as a first portion 116a and a second portion 116b. The first portion 116a may be angled relative to the second portion 116b. Similarly, the distal surface 120 may include a continuously curved surface or distinctly angled portions such as a first portion 120a and a second portion 120b. The first portion 120a may be angled relative to the second portion 120b.

When the instrument 100 is in the first position shown in FIG. 8, the first portion 116a of the proximal surface 116 and the first portion 120a of the distal surface 120 grasp the rod 200. The first portion 116a and the first portion 120a may be substantially parallel and separated by a thickness T of the rod 200. When the instrument 100 is in the second position shown in FIG. 9, the second portion 116b of the proximal surface 116 and the second portion 120b of the distal surface 120 grasp the rod 200. The second portion 116b and the second portion 120b may be substantially parallel and separated by the thickness T of the rod.

Example embodiments of the methods and systems of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

The invention claimed is:

1. An instrument for grasping and articulating a surgical implant, comprising:
a first arm having a distal end with a proximal-facing surface; and
a second arm pivotably coupled to the first arm and having a distal end with a distal-facing surface,
wherein the proximal-facing surface and the distal-facing surface grasp the implant at a first angle in a first position and a second angle in a second position.

2. The instrument of claim 1, wherein a first portion of the proximal-facing surface opposes a first portion of the distal-facing surface in the first position and a second portion of the proximal-facing surface opposes a second portion of the distal facing surface in the second position.

3. The instrument of claim 1, wherein at least one of the first arm and the second arm includes a first length in the first position and a second length in the second position.

4. The instrument of claim 1, wherein the first angle is greater than or equal to sixty degrees from a longitudinal axis of the instrument and the second angle is less than sixty degrees from the longitudinal axis of the instrument.

5. The instrument of claim 1, wherein the distal ends are offset in the first position and aligned in the second position.

6. The instrument of claim 1, wherein the proximal-facing surface and the distal-facing surface are separated by a fixed distance in the first and second positions that corresponds to a thickness of the implant.

7. The instrument of claim 1, wherein the first arm includes an adjustable length and the second arm includes a fixed length greater than the adjustable length.

8. The instrument of claim 7, wherein the adjustable length decreases from a first length to a second length as the first arm and the second arm pivot from the first position to the second position.

9. The instrument of claim 1, wherein the first arm includes a fixed length and the second arm includes an adjustable length that is greater than the fixed length.

10. The instrument of claim 9, wherein the adjustable length increases from a first length to a second length as the first arm and the second arm pivot from the first position to the second position.

11. An instrument for gripping and manipulating a spinal rod, comprising:
a fixed length arm having a distal end with first and second proximal-facing grips; and
an adjustable length arm having a distal end with first and second distal-facing grips and pivotably coupled and movable between first and second positions relative to the fixed length arm,
wherein the adjustable length arm includes a first length and the first proximal-facing grip and the first distal-facing grip grasp the spinal rod at a first angle in the first position, and
wherein the adjustable length arm includes a second length and the second proximal-facing grip and the second distal-facing grip grasp the spinal rod at a second angle in the second position.

12. The instrument of claim 11, wherein the first length is greater than the second length.

13. The instrument of claim 11, wherein the fixed length arm includes a fixed length greater than the first and second lengths.

14. The instrument of claim 11, wherein the distal ends offset in the first position and align in the second position.

15. The instrument of claim 11, further comprising a bias member that biases the adjustable length arm to the first length in the first position and compresses when the adjustable length arm decreases to the second length in the second position.

16. An instrument for gripping and manipulating a spinal rod, comprising:
a fixed length arm having a distal end with first and second distal-facing grips; and
an adjustable length arm having a distal end with first and second proximal-facing grips and pivotably coupled and movable between first and second positions relative to the fixed length arm,
wherein the adjustable length arm includes a first length and the first proximal-facing grip and the first distal-facing grip grasp the spinal rod at a first angle in the first position, and
wherein the adjustable length arm includes a second length and the second proximal-facing grip and the second distal-facing grip grasp the spinal rod at a second angle in the second position.

17. The instrument of claim 16, wherein the first length is less than the second length.

18. The instrument of claim 16, wherein the fixed length arm includes a fixed length less than the first and second lengths.

19. The instrument of claim 16, wherein the distal ends offset in the first position and align in the second position.

20. The instrument of claim 16, further comprising a bias member that biases the adjustable length arm to the first length in the first position and stretches when the adjustable length arm increases to the second length in the second position.
